(19) United States
(12) Patent Application Publication
(10) Pub. No.: US 2009/0012172 A1
(43) Pub. Date: Jan. 8, 2009

(54) Cosmetic/pharmaceutical compositions comprising retinoids and anti-irritants and treatment of keratinization disorders therewith

(75) Inventors: Laurent Fredon, Roquefort Les Pins (FR); Claire Mallard, Mougins (FR); Eve Ferrara, Valbonne (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/213,153

(22) Filed: Jun. 16, 2008

(30) Foreign Application Priority Data
Dec. 15, 2005 (FR) 0512761
Nov. 28, 2006 (FR) PCT/FR2006/051239

(51) Int. Cl. A61K 31/192; A61K 31/19; A61P 17/00; A61P 17/10

(52) U.S. Cl. 514/569; 514/557

(57) ABSTRACT

Topically applicable cosmetic/pharmaceutical compositions contain at least one retinoid and at least one anti-irritant compound selected from among the salts of 18β-glycyrrhetinic acid and derivatives thereof, and are useful for the treatment and/or prevention of a dermatological condition or affliction related to a disorder of keratinization relating to cell differentiation and to cell proliferation, e.g. acne vulgaris.

### CROSS-REFERENCE TO PRIORITY/PCT APPLICATIONS

This application claims priority under 35 U.S.C. § 119 of FR 0512761, filed Dec. 15, 2005, and is a continuation of PCT/FR 2006/051239, filed Nov. 28, 2006, and designating the United States (published in the French language on Jun. 28, 2007, as WO 2007/071860 A2; the title and abstract were also published in English), each hereby expressly incorporated by reference in its entirety and each assigned to the assignee hereof.

### CROSS-REFERENCE TO COMPANION APPLICATION

Copending U.S. patent application Ser. No. ______ [Attorney Docket No. 1034227-000925], filed concurrently herewith, hereby expressly incorporated by reference and also assigned to the assignee hereof.

### BACKGROUND OF THE INVENTION

1. Technical Field of the Invention

The present invention relates to compositions for topical administration and to their applications as cosmetic or pharmaceutical products, such compositions being useful, in particular, for the treatment of acne.

2. Description of Background and/or Related and/or Prior Art

Acne is a common multifactor pathology which affects skin rich in sebaceous glands (face, scapula region, arms and inter-triginous regions). It is the commonest form of dermatosis. The following five pathogenic factors play a determining role in the formation of acne:

1. genetic predisposition;

2. overproduction of sebum (seborrhea);

3. androgens;

4. follicular keratinization disorders (comedogenesis); and

5. bacterial colonization and inflammatory factors.

Several forms of acne exist, all having in common that the pilosebaceous follicles are attacked. Exemplary are acne conglobata, acne keloid on the back of the neck, acne medicamentosa, recurrent acne miliaria, acne necrotica, acne neonatorum, premenstrual acne, occupational acne, acne rosacea, senile acne, solar acne and acne vulgaris.

Acne vulgaris, also known as polymorphous juvenile acne, is the commonest. It comprises four stages:

Stage 1 corresponds to comedonal acne, characterized by a large number of open and/or closed comedones and of microcysts.

Stage 2, or papulopustular acne, is of mild to moderate seriousness. It is characterized by the presence of open and/or closed comedones and of microcysts but also of red papules and of pustules. It mainly affects the face and leaves few scars.

Stage 3, or papulocomedonal acne, is more serious and extends to the back, to the thorax and to the shoulders. It is accompanied by a larger number of scars.

Stage 4, or nodulocystic acne, is accompanied by numerous scars. It exhibits nodules and also large painful purplish pustules.

The various forms of acne described above can be treated with active principles, such as anti-seborrheics and anti-infectives, for example benzoperoxide (in particular the product Eclaran® marketed by Pierre Fabre), with retinoids, such as tretinoin (in particular the product Retacnyl® marketed by Galderma) or isotretinoin (product Roaccutane® marketed by Laboratoires Roche), or with naphthoic acid derivatives. Naphthoic acid derivatives, such as, in particular, 6-[3-(1-adamantyl)-4-methoxyphenyl]-2-naphthoic acid, commonly known as adapalene (the product Differin® marketed by Galderma), are widely described and recognized as active principles which are as effective as tretinoin in the treatment of acne.

Adapalene in particular exhibits an effectiveness which is accepted by all; however, it would be advantageous and useful for its tolerance by the topical route, although better than that of its competitors belonging to the same chemical category (tretinoin, tazarotene), to be improved.

### SUMMARY OF THE INVENTION

It has now surprisingly been discovered that the combination of adapalene with certain specific anti-irritant compounds significantly improves the tolerance of this retinoid and thus overcomes the problem of irritation. This is because, as shown in the Example 2 to follow, certain anti-irritants reduce by up to more than 60% the edema caused by adapalene.

The present invention thus features compositions, in particular pharmaceutical compositions and preferably dermatological compositions, intended in particular for topical application, comprising, formulated into a physiologically acceptable medium, at least one retinoid compound, preferably selected from among the naphthoic acid derivatives of formula (I) below, their salts and their esters, and at least one anti-irritant compound selected from among the salts of 18β-glycyrrhetinic acid and derivatives thereof.

Advantageously, the subject compositions do not comprise any depigmenting agent other than the retinoid compound, in particular adapalene.

The term “physiologically acceptable medium” means a medium compatible with the skin, mucous membranes and/or superficial body growths.

### BRIEF DESCRIPTION OF THE DRAWINGS

FIG. 1 is a graph comparing the irritant power of reference gels versus that according to the present invention;

FIG. 2 is a histogram of the AUC for edemas D2-D19; and

FIG. 3 is a graph showing the number of comedones on the back of Rhino mice after 18 days of topical treatment with the gels of FIG. 1.

### DETAILED DESCRIPTION OF BEST MODE AND SPECIFIC/PREFERRED EMBODIMENTS OF THE INVENTION

The retinoid compounds according to the invention can be selected from among all trans retinoic acid (or tretinoin), isotretinoin or motretinide.

The retinoid compounds according to the invention are preferably selected from among naphthoic acid derivatives of formula (I), the salts and the esters thereof:

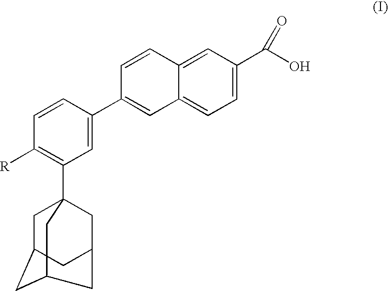

wherein R is a hydrogen atom, a hydroxyl radical, a linear or branched alkyl radical having from 1 to 4 carbon atoms, an alkoxy radical having from 1 to 10 carbon atoms or a cycloaliphatic radical which is unsubstituted or substituted.

The term “linear or branched alkyl radical having from 1 to 4 carbon atoms” means, preferably, the methyl, ethyl, propyl and butyl radicals.

The term “alkoxy radical having from 1 to 10 carbon atoms” means, preferably, the methoxy, ethoxy, propoxy, butoxy, hexyloxy and decyloxy radicals.

The term “cycloaliphatic radical” means, preferably, mono- or polycyclic radicals, such as the 1-methylcyclohexyl radical or the 1-adamantyl radical.

The term “salts of the naphthoic acid derivatives” means salts formed with a pharmaceutically acceptable base, in particular an inorganic base, such as sodium hydroxide, potassium hydroxide and aqueous ammonia, or an organic base, such as lysine, arginine or N-methylglucamine, but also the salts formed with fatty amines, such as dioctylamine, aminomethylpropanol and stearylamine.

The term “esters of the naphthoic acid derivatives” means esters formed with pharmaceutically acceptable alcohols.

Preferably, the selection will be made, among the naphthoic acid derivatives included in the compositions according to the invention, of 6-[3-(1-adamantyl)-4-methoxyphenyl]-2-naphthoic acid (adapalene), 6-[3-(1-adamantyl)-4-hydroxyphenyl]-2-naphthoic acid, 6-[3-(1-adamantyl)-4-decyloxyphenyl]-2-naphthoic acid or 6-[3-(1-adamantyl)-4-hexyloxyphenyl]-2-naphthoic acid.

More preferably still, the retinoid compounds according to the invention are selected from among adapalene (6-[3-(1-adamantyl)-4-methoxyphenyl]-2-naphthoic acid), its salts and its esters.

The term “adapalene salts” means, in particular, the salts formed with a pharmaceutically acceptable base, in particular inorganic bases, such as sodium hydroxide, potassium hydroxide and aqueous ammonia, or organic bases, such as lysine, arginine or N-methylglucamine.

The term “adapalene salts” also means the salts formed with fatty amines, such as dioctylamine, aminomethylpropanol and stearylamine.

Preferably, the retinoid compound is adapalene.

The anti-irritants according to the present invention are salts of 18β-glycyrrhetinic acid and derivatives thereof. The inclusion of these specific anti-irritants makes it possible to reduce the irritation caused by retinoids, in particular adapalene.

The term “salts of 18β-glycyrrhetinic acid” means, in particular, the potassium salt of 18β-glycyrrhetinic acid, the sodium salt of 18β-glycyrrhetinic acid, the monoammonium salt of 18β-glycyrrhetinic acid (ammonium glycyrrhetinate), the disodium salt of 18β-glycyrrhetinic acid succinate or the dipotassium salt of 18β-glycyrrhetinic acid.

The term “derivatives of the salts of 18β-glycyrrhetinic acid” means in particular the monoester of glycerol and of 18β-glycyrrhetinic acid.

Preferably, the anti-irritant is the potassium salt of 18β-glycyrrhetinic acid.

In the compositions according to the invention, the concentration of retinoid compound is from 0.001% to 10% by weight, preferably from 0.01% to 5% by weight and more preferably from 0.05% to 2% by weight of the total weight of the composition. Herein, unless otherwise specified, it is understood that, when concentration intervals are given, they include the upper and lower limits of said interval.

Preferably, the concentration of retinoid compound is equal to 0.01%. Alternatively, the concentration of retinoid compound is preferably equal to 0.3%.

The concentration of anti-irritant compound is, for its part, from 0.01% to 10%, preferably from 0.1% to 7% by weight.

The compositions according to the present invention can be provided in all the formulation forms normally employed for topical application, in particular in the form of aqueous, aqueous/alcoholic or oily dispersions, of dispersions of the lotion type, of aqueous, anhydrous or lipophilic gels, of emulsions with a liquid or semi-liquid consistency of the milk type, obtained by dispersion of a fatty phase in an aqueous phase (O/W) or vice versa (W/O), or of suspensions or emulsions with a soft, semi-liquid or solid consistency of the cream, cream gel, foam or ointment type, or of microemulsions, of microcapsules, of microparticles or of vesicular dispersions of ionic and/or nonionic type, or in the form of sprays.

Preferably, the compositions are provided in the form of a gel.

One skilled in the art will take care to select the excipients constituting the compositions according to the invention as a function of the formulation form desired and such that the advantageous properties of the composition according to the invention are retained.

In addition, the compositions according to the invention can, in particular, comprise one or more of the following ingredients:

a) one or more gelling agents or suspending agents,

b) one or more chelating agents,

c) one or more wetting agents,

d) one or more preservatives.

Exemplary gelling agents or suspending agents which can be included in the compositions according to the invention are carbomers marketed under the generic name of Carbopol®, carbomers said to be insensitive to electrolytes marketed under the trademark of Ultrez 10® or of Carbopol ETD by BF Goodrich, polysaccharides, with, as non-limiting examples, xanthan gum, such as Keltron T®, marketed by Kelco, guar gum, chitosans, cellulose and its derivatives, such as hydroxyethylcellulose, in particular the product marketed under the trademark of Natrosol HHX 250® by Aqualon, and the copolymer of sodium acrylamide and of acrylamido-2-methylpropanesulfonate as a 40% dispersion in isohexadecane, and polysorbate 80, marketed under the trademark of Simulgel 600® by Seppic.

A preferred gelling agent is hydroxyethylcellulose, marketed, in particular, under the trademark Natrosol HHX 250®.

Exemplary chelating agents include diethylenetriaminepentaacetic acid (DTPA), ethylenediaminedi(o-hydroxyphenylacetic acid) (EDDHA), (2-hydroxyethyl)ethylenediaminetriacetic acid (H EDTA), ethylenediaminedi(o-hydroxy-p-methylphenylacetic acid) (EDDHMA), ethylenediaminetetraacetic acid (EDTA) and ethylenediaminedi(5-carboxy-2-hydroxyphenylacetic acid) (EDDCHA).

Preferably, wetting agents are included, the role of which is to reduce the surface tension and to make possible greater spreading of the liquid, and exemplary thereof are compounds such as propylene glycol, dipropylene glycol, propylene glycol dipelargonate, lauroglycol and ethoxydiglycol, alone or as a mixture. Also, compounds may be included known for their role as emulsifiers, such as Tween 80, glyceryl monostearate & POE stearate, marketed under the trademark Arlacel 165FL® by Uniquema, polyoxyethylene (21) stearyl ether, marketed under the trademark Brij 721® by Uniquema, or Synperonics, with in particular Synperonic PE/L62 (poloxamer 182) or Synperonic PE/L44 (poloxamer 124).

A preferred wetting agent is propylene glycol, Synperonic PE/L62 (poloxamer 182) or Synperonic PE/L44 (poloxamer 124).

Exemplary preservatives that can be included are benzoic acid and its derivatives with benzyl alcohol, benzalkonium chloride, sodium benzoate, bronopol, chlorhexidine, chlorocresol and its derivatives, ethyl alcohol, phenethyl alcohol, phenoxyethanol, potassium sorbate, diazolidinylurea, parabens, such as propylparaben or methylparaben, taken alone or as mixtures.

Preferred preservative are the parabens and phenoxyethanol or benzalkonium chloride, alone or as mixtures.

The compositions according to the invention can also comprise one or more emulsifiers.

Surface-active emulsifiers are amphiphilic compounds which have a hydrophobic moiety possessing an affinity for the oil and a hydrophilic moiety possessing an affinity for the water, thus creating a connection from the two phases. Ionic or nonionic emulsifiers thus stabilize oil/water emulsions by being adsorbed at the interface and by forming lamellar layers of liquid crystals.

Exemplary preferred emulsifiers include the emulsifiers mentioned above for their property of wetting agents or lipophilic emulsifiers of glucate SS and glucamate SSE type.

The compositions of the invention can additionally comprise any additive normally used in the cosmetics or pharmaceutical field, such as neutralizing agents, sunscreens, antioxidants, fillers, electrolytes, colorants, normal inorganic or organic bases or acids, fragrances, essential oils, cosmetic active principles, moisturizing agents, vitamins, essential fatty acids, sphingolipids, self-tanning compounds, such as DHA, soothing and skin-protecting agents, propenetrating agents or a mixture of these. Of course, one skilled in the art will take care to choose this or these optional additional compounds and/or their amounts such that the advantageous properties of the composition according to the invention are not, or not substantially, detrimentally affected.

These additives can be present in the composition in a proportion of 0.001% to 20% by weight, with respect to the total weight of the composition.

The present invention also features administration of the subject compositions as described above as medicaments.

In particular, this invention features the formulation of the subject compositions as described above into medicaments useful for the treatment and/or prevention of dermatological conditions or afflictions related to a disorder of keratinization relating to cell differentiation and to cell proliferation, in particular in treating acne vulgaris, comedonal acne, papulopustular acne, papulocomedonal acne, nodulocystic acne, acne conglobata, acne keloid of the back of the neck, recurrent acne miliaria, acne necrotica, acne neonatorum, occupational acne, acne rosacea, senile acne, solar acne and acne medicamentosa.

Preferably, the present invention features formulation of a composition as described above into medicaments useful to prevent and/or treat acne vulgaris, whether regime or regimen.

Preferably, said compositions according to the invention are administered topically.

In addition, this invention also features the cosmetic application of the subject compositions in the treatment of skin having a tendency toward acne, in order to combat the greasy appearance of the skin or hair.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Solutions of Anti-Irritants

The anti-irritants used are formulated, unless otherwise indicated, in an ethanol/water (50:50) vehicle at the concentrations shown in the table below. The latter also shows, for each anti-irritant, the group treated in Example 2.

| | | Content in 50/50 |
|---|---|---|
| | | aqueous/alcoholic |
| Group | Anti-irritants | solution (%) |
| 4 | 18β-Glycyrrhetinic acid (enoxolone) | 1.5 |
| 5 | Potassium salt of 18β-glycyrrhetinic acid | 1.5 |

Group 4 is used as a negative control in the studies which follow. This is because, as shown in the following studies, although being known as an anti-irritant, enoxolone does not have an effect on the irritation due to retinoids.

### EXAMPLE 2

### Evaluation of the Effects of Various Anti-Irritants in Preventive Treatment Before Topical Application of Differin® Gel on the BALB/c Mouse; Tolerance Study

The goal of the present study is to compare the irritant power of a reference gel, comprising 0.1% adapalene, when this treatment is preceded or not preceded by treatment with an anti-irritant.

The treatment consists of a daily topical application (20 μl) of anti-irritant, formulated in an aqueous/alcoholic vehicle (50% ethanol and 50% water by volume), on the internal face of the right ear of BALB/c mice divided into 15 groups (female mice approximately 9 weeks old), followed by a topical application (20 μl) of Differin® gel (reference gel comprising 0.1% adapalene) at the rate of one application of each formulation per day for 6 days.

The test products are:

Group 1: Untreated (controls)

Group 2: Differin® gel (reference gel)

Group 3: Ethanol/water solution (aqueous/alcoholic vehicle for the anti-irritants), followed by Differin® gel

Group 4: Enoxolone, followed by Differin® gel

Group 5: Potassium salt of 18β-glycyrrhetinic acid, followed by Differin® gel

Evaluation is carried out by measurements of the thickness of the ear by means of the Oditest and by clinical observation of the animals from the 2^{nd} to 19^{th} day.

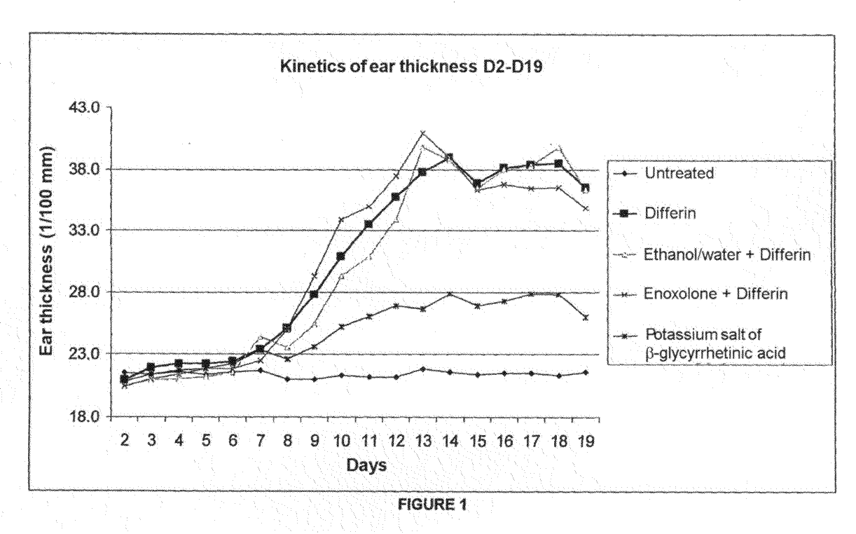

The results are represented in the table below and in FIG. 1:

FIG. 1 is the kinetics of the mean thickness of the mouse ears from the 2^{nd} and 19^{th} days for groups 1 to 3 (references) and 4 and 5. These kinetics show that the Differin® gel formulation alone is an irritant; the addition of the aqueous/alcoholic vehicle to this formulation does not change the degree of irritation (group 3).

Enoxolone (group 4) has virtually no effect on the decrease in the irritation caused by Differin® gel.

On the other hand, the application of the potassium salt of 18β-glycyrrhetinic acid (group 5) significantly reduces the irritation caused by Differin® gel; this decrease is greater than 60%.

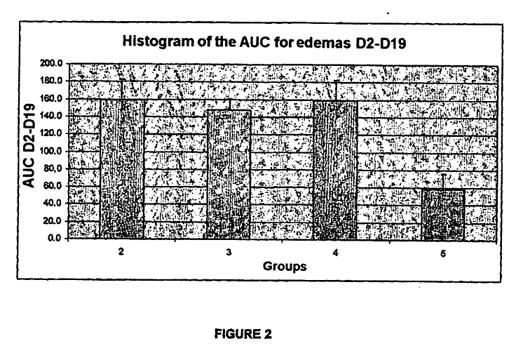

Summarizing table for the results of the areas under the curve (AUC) for the kinetics of the ear thicknesses (cf. FIG. 2 for the histograms)

| | AUC for | | % AUC | | |
|---|---|---|---|---|---|
| | edema | | inhibition | | Student's |
| | D2-D19 | | vs. | p | t-test vs. |
| | Mean | SEM | Differin | values | Differin |
| Untreated | | | | | |
| Differin | 158.6 | 24.4 | | | |
| Ethanol/water + Differin | 147.6 | 12.8 | 7.0 | 0.6989 | NS |
| Enoxolone + Differin | 159.0 | 23.8 | −0.3 | 0.9909 | NS |
| Potassium salt of 18β- | 58.2 | 17.5 | 63.3 | 0.0102 | * |
| glycyrrhetinic acid + | | | | | |
| Differin | | | | | |

NS = Not Significant

The results of the study show that, after repeated topical applications of 20 μl of an anti-irritant solution, followed by 20 μl of Differin® gel, from D1 to D6 on the ear of the BALB/c mouse:

the Differin® gel formulation is an irritant;

enoxolone does not have an effect on the irritation caused by Differin® gel;

on the other hand, the anti-irritant potassium salt of 18β-glycyrrhetinic acid reduces the edema by 63%.

This example shows that potential anti-irritants do not all have the same effect with regard to the edema caused by Differin® gel and that only the potassium salt of 18β-glycyrrhetinic acid is effective in reducing the irritation due to adapalene.

It should also be noted that no loss in weight is recorded during the study.

### EXAMPLE 3

### Evaluation of the Comedolytic Activity of Differin® Gel Alone Compared with Differin® Gel in Combination with a Placebo Comprising an Anti-Irritant on the Rhino Mouse

The goal of the present study is to compare the comedolytic activity of Differin® gel (reference gel comprising 0.1% adapalene) whether this treatment is preceded or not preceded by a treatment with an anti-irritant.

The treatment consists of a daily topical application of an aqueous/alcoholic vehicle (ethanol/water 50:50) comprising the anti-irritant potassium salt of 18β-glycyrrhetinic acid, followed 30 minutes later by an application of Differin® gel, on the skin of the back of the RHINO FVB/N RJ-hr^{th} (Rhino) mouse for 18 days.

The test products are:

Group 1: Differin® gel alone

Group 2: Aqueous/alcoholic vehicle, followed by Differin® gel placebo

Group 3: Aqueous/alcoholic vehicle, followed by Differin® gel

Group 4: Potassium salt of 18β-glycyrrhetinic acid, followed by Differin® gel.

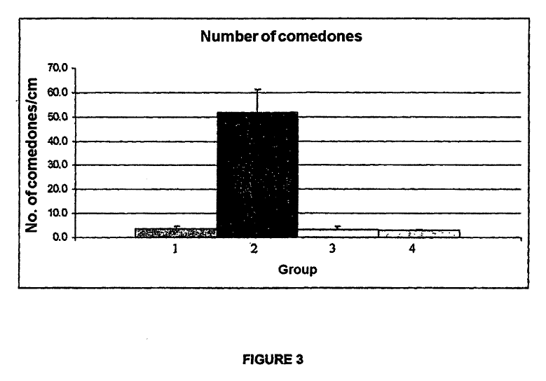

FIG. 3 shows the results of the counting of the number of comedones per centimeter [cm] on the back of the Rhino mice after 18 days of topical treatment for the 4 groups mentioned above.

The results of the study show that skin treated with placebos (group 2) exhibits a high number of comedones per centimeter, of from 51 and 60. Skin treated with Differin® gel alone or preceded by a placebo (groups 1 and 3) exhibits a comparable and low number of comedones per centimeter, of from 3 and 5.

Skin treated beforehand with the anti-irritant potassium salt of 18β-glycyrrhetinic acid exhibits a number of comedones per centimeter statistically equivalent to that of groups 1 and 3.

It thus emerges from FIG. 3 that the anti-irritant potassium salt of 18β-glycyrrhetinic acid with Differin® gel in a split treatment does not reduce the comedolytic activity of Differin® gel alone.

Finally, after 18 days of topical treatment, it should be noted that the animals do not exhibit a loss in weight.

This overall study demonstrates that the application of the specific potassium salt of 18β-glycyrrhetinic acid before the treatment with Differin® gel does not reduce the comedolytic activity of Differin® gel.

### EXAMPLE 4

### Formulations of Gel Type Comprising 0.1% Adapalene and Anti-Irritants

| Ingredients | Formula A |
|---|---|
| Adapalene | 0.1% |
| Purified water | 80.0% |
| Potassium salt of 18β-glycyrrhetinic acid | 1.5 |
| Titriplex III | 0.2% |
| Natrosol 250 HHX Pharm | 2.0% |
| Propylene glycol | 4.0% |
| Synperonic PE/L62 | 0.2% |
| Phenoxyethanol | 1.0% |
| Purified water | q.s. for 100% |

### EXAMPLE 5

### Study of Tolerance of the Formulations of Example 4

A study of tolerance is carried out according to the protocol of Example 2 with the formulation of Example 4. However, the present case, in contrast to Example 2, relates to a treatment which is not split since the adapalene and the anti-irritant are present in the same formulation.

The results of the areas under the curve (AUC) for the kinetics of ear thickness from days 2 and 19 are reported in the following table:

| | | | % | | |
|---|---|---|---|---|---|
| | AUC D2-D19 | | Increase | Student's | % Inhibition |
| | | Standard | vs. | t-test | vs. Differin |
| | Mean | deviation | placebo | vs. placebo | gel |
| Differin | 358.0 | 3.8 | | | |
| placebo | | | | | |
| Differin gel, | 519.5 | 25.8 | 45.1 | *** | |
| 0.1% | | | | | |
| Formula A | 402.4 | 17.0 | 12.1 | * | 22.5 |

Conclusions of the study:

0.1% Differin® gel increases the area under the curve by 45% with respect to the placebo gel.

The formulation with the anti-irritant increases the area under the curve with respect to the placebo gel by 12%.

With respect to 0.1% Differin® gel, formula A is less irritating by 20%.

The potassium salt of 18β-glycyrrhetinic acid appears to be an effective anti-irritant. These results confirm those of Example 2, where the anti-irritant had been evaluated in the split treatment, namely, before application of 0.1% Differin® gel.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/pharmaceutical composition comprising at least one retinoid compound selected from the group consisting of all trans retinoic acid, isotretinoin, motretinide, naphthoic acid compounds having the structural formula (I) and the salts and esters thereof: 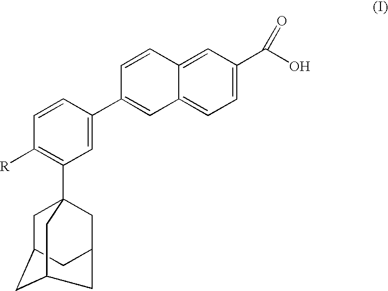
where R is a hydrogen atom, a hydroxyl radical, a linear or branched alkyl radical having from 1 to 4 carbon atoms, an alkoxy radical having from 1 to 10 carbon atoms or a substituted or unsubstituted cycloaliphatic radical, and at least one anti-irritant compound selected from the group consisting of salts of 18β-glycyrrhetinic acid and derivatives thereof, formulated into a topically applicable, physiologically acceptable medium therefor.

2. The cosmetic/pharmaceutical composition as defined by claim 1, wherein said at least one retinoid has the formula (I) and R is a methyl, ethyl, propyl or butyl radical.

3. The cosmetic/pharmaceutical composition as defined by claim 1, wherein said at least one retinoid has the formula (I) and R is a methoxy, ethoxy, propoxy, butoxy, hexyloxy or decyloxy radical.

4. The cosmetic/pharmaceutical composition as defined by claim 1, wherein said at least one retinoid has the formula (I) and R is the 1-methylcyclohexyl radical or the 1-adamantyl radical.

5. The cosmetic/pharmaceutical composition as defined by claim 1, wherein said at least one retinoid compound is selected from the group consisting of adapalene, 6-[3-(1-adamantyl)-4-hydroxyphenyl]-2-naphthoic acid, 6-[3-(1-adamantyl)-4-decyloxyphenyl]-2-naphthoic acid and 6-[3-(1-adamantyl)-4-hexyloxyphenyl]-2-naphthoic acid.

6. The cosmetic/pharmaceutical composition as defined by claim 1, wherein said at least one retinoid compound is adapalene.

7. The cosmetic/pharmaceutical composition as defined by claim 1, wherein said at least one anti-irritant compound is the potassium salt of 18β-glycyrrhetinic acid.

8. The cosmetic/pharmaceutical composition as defined by claim 1, formulated as a gel.

9. The cosmetic/pharmaceutical composition as defined by claim 1, said at least one retinoid compound comprising from 0.001% to 10% by weight of the total weight thereof.

10. The cosmetic/pharmaceutical composition as defined by claim 9, wherein the concentration of said at least one retinoid compound is about 0.1%.

11. The cosmetic/pharmaceutical composition as defined by claim 9, wherein the concentration of said at least one retinoid compound is about 0.3%.

12. The cosmetic/pharmaceutical composition as defined by claim 1, wherein the concentration of said at least one anti-irritant compound ranges from 0.01% to 10% by weight of the total weight thereof.

13. The cosmetic/pharmaceutical composition as defined by claim 1, formulated as a medicament.

14. A regime or regimen for the treatment and/or prevention of a dermatological condition or affliction related to a disorder of keratinization relating to cell differentiation and to cell proliferation, comprising topically applying onto the affected skin area of an individual in need of such treatment, a thus effective amount of the cosmetic/pharmaceutical composition as defined by claim 1.

15. A regime or regimen for the treatment of acne vulgaris, comedonal acne, papulopustular acne, papulocomedonal acne, nodulocystic acne, acne conglobata, acne keloid of the back of the neck, recurrent acne miliaria, acne necrotica, acne neonatorum, occupational acne, acne rosacea, senile acne, solar acne or acne medicamentosa, comprising topically applying onto the affected skin area of an individual in need of such treatment, a thus effective amount of the cosmetic/pharmaceutical composition as defined by claim 1.

16. The regime or regimen as defined by claim 15, comprising the treatment and/or prevention of acne vulgaris.

17. A regime or regimen for the treatment of skin having a tendency toward acne or for combating the greasy appearance of the skin or hair, comprising topically applying onto the affected skin area of an individual in need of such treatment, a thus effective amount of the cosmetic/pharmaceutical composition as defined by claim 1.
